(19) United States
(12) Patent Application Publication
(10) Pub. No.: US 2003/0148518 A1
(43) Pub. Date: Aug. 7, 2003

(54) Method for mass propogation of podophyllum hexandrum royle using embryo culture technique

(76) Inventors: Amit Chandra Kharkwal, Palampur (IN); Om Prakash, Palampur (IN); Amita Bhattacharya, Palampur (IN); Paramvir Singh Ahuja, Palampur (IN)

Correspondence Address: JENKENS & GILCHRIST, P.C., 225 WEST WASHINGTON, SUITE 2600, CHICAGO, IL, 60606, US

(21) Appl. No.: 09/815,126

(22) Filed: Mar. 22, 2001

(51) Int. Cl. C12N 5/00; C12N 5/02

(52) U.S. Cl. 435/430.1

(57) ABSTRACT

The present invention describes an improved method of Podophyllum hexandrum Royle through embryo culture technique by using B5 medium supplemented with 3% sucrose and 0.8% agar under initial dark conditions for embryo germination and initial embling growth followed by light/dark photoperiod regime for further embling development.

### FIELD OF THE INVENTION

The present invention relates to an improved method for mass propagation of Podophyllum hexandrum Royle using embryo culture technique.

### BACKGROUND OF THE INVENTION

Podophyllum hexandrum Royle is an important medicinal plant, which grows in the inner ranges of Himalayas at an altitude of 2700-4200 m from Kashmir to Sikkim (India), (Badhawar and Sharma 1963), and extending up to south west of China.

The rhizomes of Podophyllum hexandrum are rich in podophyllotoxins and have anticancer and antitumor properties (Kamil and Dewick, 1986). The podophyllotoxins are lignans whose semi-synthetic derivatives—etoposide (VP-16-213) and teniposide (VM-26) are approved drugs for the treatment of testicular and lung cancer (Stahelin and Warburg, 1991). P. hexandrum has the maximum podophyllotoxins up to about 4% from dried roots as compared to 0.25% of P. peltatum (Jackson and Dewick, 1984a).

Ever increasing demand for this drug in modern medicine, coupled with its existing use in traditional system of medicine has resulted in ruthless uprooting of the under ground parts of the plant leading to intense collection coupled with the lack of organized cultivation. Consequently P. hexandrum has been declared as an endangered species (Airi et al. 1997, Bhadula et al. 1997) thus expeditious methods are required for its sustained propagation and organized utilization. Moreover since Podophyllum generally grows at high altitudes of the Himalayas and inaccessible locales, it is all the more essential to cultivate these plants in large numbers (Badhawar and Sharma, 1963) for its sustainable utilization.

Early attempts in domestication of Podophyllum were through multiplication of rhizomes however, since rhizomes are also the source of podophyllotoxins, considerable loss is incurred either in terms of propagules (when uprooted) or important harvestable materials of pharmaceutical importance (when conserved) (Sadowska et al. 1997). The other infrequent method of propagation is through seed germination. Seed germination is erratic and is further limited by long periods of seed coat imposed or mechanical dormancy and endosperm dormancy which cannot be generally broken by traditional methods (Badhawar and Sharma, 1963). More than the germinability of the seeds, the structural barrier in the seedlings i.e. the hypocotyl (Purohit and Nautiyal 1986) may also reduce the chances of its survival in nature (Nautiyal et al. 1987).

Thus, it is not surprising that despite the urgent necessity of mass propagation and conservation of Podophyllum, the work done till date has been limited only to academic interests with respect to the small number of plants propagated. The major objective in the propagation of endangered species is conservation of valuable heterogeneity. Embryo culture technique seems to be a useful approach in this regard as this technique not only offers a method to circumvent the problems of mechanical and endosperm dormancy (Collins and Groccer, 1984) but also helps in producing a large population of heterogeneous plants, representative of the entire gene pool.

The present invention relates to a method which is is not only time effective but also reproducible for the large scale production of P. hexandrum plants through embryo culture.

Reference may be made to the work of Arumugam and Bhojwani, 1989 wherein embryos were cultured on MS medium supplemented with 3% sucrose and more than 90% germination was achieved after 7 days under a photoperiod of 14 h light and 10 h dark. The drawbacks are that even though some of the plantlets were transferred to the field, no fresh growth was recorded even after four weeks, also even after 3 repetitive subcultures of 1 month each true leaf emergence was not recorded. Recently Nadeem et al (2000) also used embryo culture technique for propagation on MS medium supplemented with 3% sucrose and achieved 100% germination and successful embling growth. However, the drawbacks are no field transfer and true leaf emergence was achieved by them. Reference may be made to the related species Podophyllum peltatum (Sadowska et al. 1997) wherein excised zygotic embryos were germinated on basal MS medium supplemented with 3% sucrose and 1 mg/liter Gibberellic acid. However, the drawback of this technique is that only 34% of the embryos germinated after one week, out of which only 31% developed into plantlets after 5 weeks and there was no mention of field transfer.

### OBJECTS OF THE INVENTION

The main object of the present invention is to provide an improved method for mass propagation of Podophyllum hexandrum Royle using embryo culture technique which obviates the drawbacks.

Another object is to provide an improved method for mass propagation of Podophyllum hexandrum Royle using embryo culture technique.

Yet another object is to raise heterogeneous population of Podophyllum hexandrum Royle as to maintain the maximum genetic diversity which may be below the species level.

Still another object is to transfer the ex situ raised plants to the in situ conditions.

Yet another object is to provide a uniform protocol for field transfer of in vitro raised Podophyllum hexandrum plants.

Yet another object is to shorten the life cycle significantly.

Yet another object is to specify nutritional requirement for Podophyllum hexandrum propagation under in vitro conditions.

Yet another object is to specify the potting mix required at various stages of field transfer.

Yet another object is to specify the growth conditions for the field transferred plants of Podophyllum hexandrum.

Yet another object is to provide characterized planting materials.

### SUMMARY OF THE INVENTION

The present invention provides an improved method for mass propagation of Podophyllum hexandrum using embryo culture technique.

### DETAILED DESCRIPTION OF THE INVENTION

Accordingly the invention provides an improved method for mass propagation of Podophyllum hexandrum Royle using embryo culture technique, said method comprising the steps of:

(a) placing an embryogenic tissue in a culture initiation medium comprising B-5 vitamins, supplemented with sucrose and agar and having pH of 5.8, at a temperature between 20 to 40° C. and subjected to a light/dark regime for about one week, to obtain embryos with radicals and expanded cotyledons,

(b) transferring the embryos to a basal MS medium supplemented with vitamins in the presence of about 14 hours of white fluorescent light of intensity of 52 [mol m⁻² s⁻¹, 10 hr dark period for 4 weeks to obtain emblings with well developed root, hypocotyl and green cotyledonary leaves,

(c) growing the emblings to acclamatize the plantlets for one week in bottles containing sand-soil and farm yard manure mix, B5 vitamins, without sucrose at a temperature between 20 to 40° C., light-dark regime and photo-period with light of intensity of 52 lmol m² s , and

(d) transferring the acclimatized plantlets to field to obtain healthy young plants with true leaves in 12 weeks.

To describe in detail, the method comprises collection of mature pods of P. hexandrum Royle from their natural habitats in the Western Himalayas at an altitude ranging from 2700 m-3500 m above mean sea level during the last week of August and first week of September. Immediately after collection, the seeds were separated from the pulp. Prior to disinfection with 0.1% Bavistin and streptomycin sulphate (0.1%) for 15 minutes, followed by a thorough washing of seeds. After a 1 minute dip in 70% alcohol and surface sterilization in 0.1% mercuric chloride for 7 minutes, all traces of mercuric chloride were finally washed off by 4-5 rinses in sterile distilled water. The surface sterilized seeds were then dissected by a sharp excision with a scalpel blade at the base of the microplyar lobe and embryos were carefully squeezed out of their micropylar groove. The embryos were cultured on B-5 (Gamborg et al., 1968) medium supplemented with 3% sucrose of pH 5.8 adjusted with 1N KOH and HCl prior to the addition of agar (0.8%) and were finally sterilized at 21° C. for 20 minutes. All the cultures were maintained in this medium for one week at 25±2° C. in dark for embryo germination. One week after germination, the cultures had fully developed radicals and expanded cotyledons which were finally transferred for further growth to 14-h light photoperiod of 52 μmolm⁻²s⁻¹ alternating with 10-h dark period. The four weeks later, the emblings (8.0 cm long) with well developed root, hypocotyl and green cotyledonary leaves were first hardened in jam bottles (covered with lids) containing 120 g of a sand: soil mix in the ratio: 3:1, enriched with 0.6 N (liquid) of B5 medium without sucrose. The emblings were hardened and grown for 1 week in culture under lab conditions (25±2° C.) and 14 h light photoperiods of 52 μmolm⁻²s⁻¹ alternating with 10-h dark period. After this, the jam bottles containing the emblings were transferred to green house, and allowed to further harden for another one week and with partially opened lids. Thereafter, the hardened emblings (8.0 cm long) were transferred to a potting mix of sand: soil: farm yard manure in the ratio: 3:1:1 for establishment into healthy young plants with true leaves within 12 weeks.

The above method has been found repeatable for 2 successive years.

In an embodiment of the present invention Podophyllum hexandrum seeds from different regimes of Himachal Pradesh, India were used.

In another embodiment of the present invention all the media were supplemented with 2.5 μM Gibberellic acid.

In another embodiment of the present invention, photoperiod regime is 14 hrs light, 10 h dark and light intensity is 52 μmolm⁻²s⁻¹.

In another embodiment, culturing the excised embryos is carried out on B5 medium under dark conditions at 24 to 29° C. for one week and the emblings with radicle, hypocotyl and expanded cotyledons are transferred after 2 weeks to light/dark photoperiod regime (14 hrs light/10 h dark).

In yet another embodiment of the present invention different media were used for embryo germination.

In still another embodiment, full and half strength B5, full and half strength MS and full and half strength WPM under different light and dark conditions were used for maximum germination.

In yet another embodiment, BS was selected as the most suitable medium out of a range of media like BS, MS and WPM with their fall and half strengths.

A major difference between the three media is the relatively high concentration of most mineral salts in MS as compared to WPM and BS (Table-1).

TABLE 1
Comparative ionic concentrations of basal MS, B5 and WPM media

| Ions | MS | B-5 | WPM |
|---|---|---|---|
| NH4 [symbol] | 20.61 mM | 2.02 mM | 4.94 mM |
| K⁺ | 20.04 mM | 24.7 mM | 12.61 mM |
| Ca⁺⁺ | 2.99 mM | 1.02 mM | 3.0 mM |
| Mg [symbol] | 1.50 mM | 1.01 mM | 1.50 mM |
| Mn [symbol] | .132 mM | .059 mM | .132 mM |
| Zn [symbol] | .029 mM | .007 mM | .030 mM |
| Na [symbol] | .224 mM | 1.10 mM | .224 mM |
| Fe⁺⁺ | .100 mM | .050 mM | .100 mM |
| NO³⁻ | 39.4 mM | 24.7 mM | 9.64 mM |
| SO4⁻ | 1.76 mM | 2.04 mM | 7.44 mM |
| PO4⁻ | 1.25 mM | 1.10 mM | 1.25 mM |
| BO3⁻⁻ | .100 mM | .049 mM | .100 mM |
| Cl⁻ | 6.0 mM | 2.04 mM | 1.31 mM |
| Fe-EDTA⁺⁺ | .110 mM | .083 mM | .110 mM |
| Co [symbol] | .105 mM | .110 mM | — |
| Cu [symbol] | .100 mM | .100 mM | .100 mM |
| MoO [symbol] | 1.03 mM | 1.03 mm | 1.03 mM |
| I⁻ | 5.0 mM | 4.52 mm | — |
| NO³⁻/NH⁴⁺ | 1.91 mM | 12.22 mM | 1.95 mM |

For improved germination 2.5, 5.0 and 7.5 μM Gibberellic acid was added to all the mediums used. Conventional MS and WPM media were appropriately modified for culture of Podophyllum embryo germination. As an example, MS and WPM media may suitably be modified as under:

| MS Medium Composition | | WPM: Medium Composition | |
|---|---|---|---|
| Major Elements | | Major Elements | |
| NH₄NO₃ | 1650 mg/litre | NH₄NO₃ | 400 mg/litre |
| KNO₃ | 1900 mg/litre | Ca(NO₃)₂.4H₂O | 556 mg/litre |
| CaCl₂.2H₂O | 440 mg/litre | K₂SO₄ | 990 mg/litre |
| MgSO₄.7H₂O | 370 mg/litre | MgSO₄.7H₂O | 370 mg/litre |
| KH₂PO₄ | 170 mg/litre | CaCl₂.2H₂O | 96 mg/litre |
| Minor Elements | | KH₂PO₄ | 170 mg/litre |
| Na₂MoO₄.2H₂O | 0.25 mg/litre | Minor Elements | |
| H₃BO₃ | 6.2 mg/litre | ZnSO₄.7H₂O | 8.6 mg/litre |
| CoCl₂.5H₂O | 0.025 mg/litre | MnSO₄.2H₂O | 22.3 mg/litre |
| CuSO₄.5H₂O | 0.025 mg/litre | CuSO₄.5H₂O | 0.25 mg/litre |
| ZnSO₄.7H₂O | 8.6 mg/litre | H₃BO₃ | 6.2 mg/litre |
| MnSO₄.2H₂O | 22.3 mg/litre | Na₂MoO₄ | 0.25 mg/litre |
| KI | 0.83 mg/litre | Na₂EDTA | 37.3 mg/litre |
| Na₂EDTA | 37.25 mg/litre | FeSO₄.7H₂O | 27.8 mg/litre |
| FeSO₄.7H₂O | 27.85 mg/litre | Vitamins | |
| Vitamins | | Inositol | 100 mg/litre |
| Glycine | 2 mg/litre | Threonine HCl | 1 mg/litre |
| Inositol | 100 mg/litre | Nicotinic Acid | 0.5 mg/litre |
| Thymine HCl | 1 mg/litre | Pyridoxine HCl | 0.5 mg/litre |
| Nicotinic Acid | 0.5 mg/litre | Glycine | 0.2 mg/litre |
| Pyridoxine HCl | 0.5 mg/litre | | |

Both light and dark conditions were tested for embling growth. Different potting mixes comprising of sand soil and farm yard manure in the ratio 3:1:1, 2:1:1 and 1:1:1, 1:3:1, 1:2:1 were tested. The different stages of emblings and their transfer were optimized for identifying the right stage and time of transfer. In another embodiment, the composition of potting mix has been specified to be 3:1 of sand :soil with 0.6N B5 medium without sucrose and a mix of sand: soil:farm yard manure in the ratio of 3:1:1for Pododphyllum plant growth. The other related species like Podophyllum peltatum can also be propagated by the method of the invention.

In another embodiment, green house conditions for successful plant establishment and true leaf emergence was selected out of the use of poly tunnels covered with and without green agro net and planting in Hikkotrays, pots and directly into potting mixes in dug trenches. The ex situ raised plants can be transferred to their natural habitat

(i) In the present method, B5 medium was chosen as the best medium as compared to all the other media for maximum germination and best embling development.

(ii) The present method defines the stage specific requirements i.e. dark for germination and early embling development at (25±2° C.) and for later embling development under dark/light photoperiod regime of 14 hours of light at 52 μmolm⁻²s⁻¹ alternating with 10-h dark period also at (25+2° C.).

(iii) This method also describes the type and concentration of specific mineral ions and NO^{3-l : NH}⁴⁺ ratio for Podophyllum germination.

(iv) This method specifies the transfer of at least 8.0 cm long emblings to covered jam bottles containing sand soil mix in the ratio 3:1 with 0.6 N B5 medium without sucrose under culture lab conditions and then their transfer from culture laboratory condition to green house condition after one week with partially opened lids. It also specifies the transfer of emblings after one week to 4″ pots containing a potting mix of sand: soil: farm yard manure in the ratio of 3:1:1. The method also specifies that the emblings in the pots should be covered with magenta jars for 6 weeks after which the magenta jars should be removed.

(v) This invention also proposes a standardized method for mass propagation of in vitro raised plants in the field.

(vi) This invention also specifies the greenhouse conditions (30° C. duing day alternating with 20° C. during night with 60-70% relative humidity) required for plant establishment, true leaf emergence and healthy growth of plants.

(vii) This invention also specifies the transfer of greenhouse grown plants to their natural habitat after hardening.

B5 despite having the lowest concentration of most mineral salts has the highest concentration of K⁺, Na⁺, and Co⁺⁺ ions and is also richer than MS in SO⁴⁻⁻ and than WPM in NO³⁻ and Cl⁻ ions. The ionic concentration of B5 medium is crucial for the embryos of Podophyllum in culture. The concentration of K⁺, Na⁺ and Co⁺⁺ is also crucial for the growth of embryos of Podophyllum culture. For instance, the concentration of K+ions may be 22 to 25 mM and that of Na⁺ ions may be 0.5 to 1.5 mM. The concentration of Co⁺⁺ ions may be 0.107 to 0.120. Further, the ratio of NO₃⁻ to NH₄⁺ ions is also crucial for Podophyllum culture. This may be in the range of 10 to 15 mM. B5 has the poorest concentration of NH⁴⁺: NO³⁻ ratio (0.081) but the ratio of NO³⁻: NH⁴⁺ is higher than WPM (1.95) and MS (1.91). Since Podophyllum has specific requirements for both the type and concentration of ions for its germination of which NO³⁻ or NO³⁻: NH⁴⁺ ratio is the most crucial. As B5 medium meets these requirements it was chosen for Podophyllum from amongst all the media An appropriate B5 medium for Podophyllum culture was modified and developed by the applicants. Such a B5 medium may comprise:

B-5: Medium Composition:

| | |
|---|---|
| Major Elements | |
| CaCl₂.2H₂O | 150 mg/litre |
| KNO₃ | 2500 mg/litre |
| MgSO₄.7H₂O | 250 mg/litre |
| (NH₄)₂SO₄ | 134 mg/litre |
| NaH₂PO₄.2H₂O | 150 mg/litre |
| Minor Elements | |
| KI | .75 mg/litre |
| H₃BO₃ | 3.0 mg/litre |
| MnSO₄.2H₂O | 10.0 mg/litre |
| ZnSO₄.7H₂O | 2.0 mg/litre |
| Na₂MoO₄.2H₂O | 0.25 mg/litre |
| CuSO₄.5H₂O | 0.025 mg/litre |
| CoCl₂.6H2O | 0.025 mg/litre |
| Na₂EDTA | 37.3 mg/litre |
| FeSO₄.7H₂O | 27.8 mg/litre |
| Vitamins | |
| Inositol | 100 mg/litre |
| Thymine HCl | 2 mg/litre |
| Glycine | 10 mg/litre |

Nitrates and its ratio with ammonium are important because it regulates different aspects of plant metabolism like nitrate assimilation into amino acids, proteins and other nitrogen containing plant constituents (Scheibe et al, 1997; Stitt and Krapp, 1999). It also regulates the increase in the levels of organic acids (Martinoia and Rentsch, 1994), in mobilization of starch and thus in its decreased levels (Scheibe et al. 1997a) and also in seed germination (Hilhorst and Karssen, 1989) by regulating the osmoticum (Stitt and Krapp, 1999) and water uptake capacity (McIntyre, 1997). Nitrates apart from being a resource also acts directly or indirectly in triggering signals that modulate gene expresson, metabolism and development (Redenbaugh and Campbell, 1991, Crawford, 1995; Stitt and Scheibe, 1998). Uptake of nitrate is probably crucial for Podophyllum embryos and this gets hindered when high concentration of ammonium is present along with nitrate or when nitrate is not the sole nitrogen source (Huang et al. 1996).

(i) It is the most efficient method for the highest percentage of field establishment and survival of P hexandrum emblings.

(ii) It also minimizes the time period for seedling establishment from 11 months 4 years (Rust and Roth 1981, Singh et al., 1999) to 4 months only.

(iii) A reproducible and defined method for mass scale germination of cultured P. hexandrum embryos as compared to the above reports.

(iv) A reproducible and defined method for mass scale germination of cultured P. hexandrum embryos as compared to the above reports which can also work in related species like Podophyllum peltatum.

(v) Light is not required for germination of the embryos as compared to the above reports

(vi) A defined method in which the light and dark requirements have been specified in terms of embryo germination, radicle emergence, hypocotyl elongation and cotyledon expansion along with growth of the healthy emblings.

(vii) The method ensures successful field establishment coupled with very low mortality rate.

(viii) The method expedites the process of true leaf emergence to the extent of 63% within 90 days. This is in contrast to the above reports wherein no true leaf emergence is achieved.

(ix) This method by way of expediting the process of true leaf emergence ensures shortening the life cycle of plant by at least one season (8 months) as compared to what happens in nature.

(x) The present method ensures statistically significant results for large-scale propagation of Podophyllum hexandrum as compared to all above reported ones.

### BRIEF DESCRIPTION OF THE ACCOMPANYING DRAWINGS

In the drawing (s) accompanying this specification:

FIG. 1 represents cultured zygotic embryos that has been excised from the seeds.

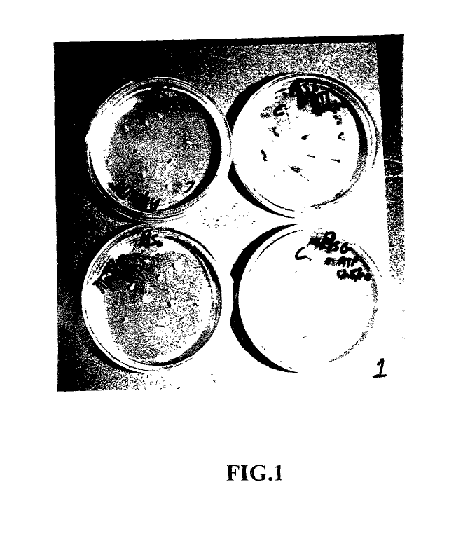

FIG. 2 represents in vitro raised emblings growing on the basal medium.

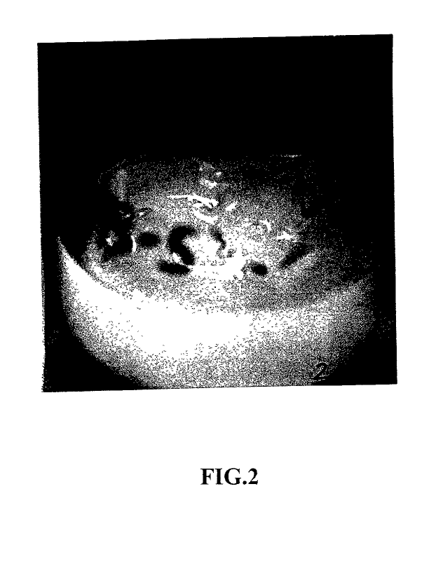

FIG. 3 represents field-transferred embling growing in the soil mix under green house condition.

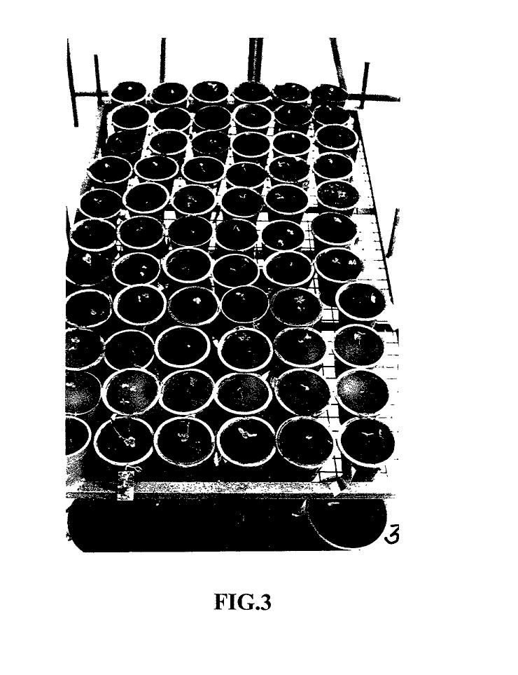

FIG. 4 represents a number of plants with true leaves growing under green house condition.

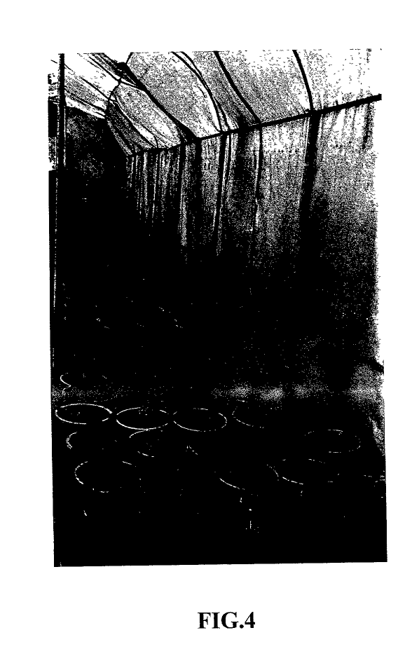

The following examples are given by way of illustration and therefore should not be construed to limit the scope of the present invention.

### EXAMPLE-1

Zygotic embryos were excised and cultured on B5 medium under dark conditions at 25±2° C. After 7 days, the embryos germinated under dark condition. After 14 days, the emblings with radicle, hypocotyl and expanded cotyledons were transferred to light/dark regime photoperiod regime (14 h light/10 h dark). After 4 weeks, the developed emblings (at least 8.0 cm long) were transferred to covered jam bottles containing 3:1:1 mixture of sand: soil: farm yard manure containing 0.6N B5 medium without sucrose. Finally, the emblings were transferred after 1 week to 4″ pots containing the above potting mix and the plants were covered with majenta jars initially for two months under green house condition. True leaves started emerging after 42 days and this continued to increase up to 90 days. After one year healthy plants with true leaves were transferred to the field.

### EXAMPLE-2

Seeds collected from different regimes of Himachal Pradesh and excised zygotic embryos were inoculated on the B5 medium and the above protocol was repeated.

### EXAMPLE-3

Excised zygotic embryos of different regimes were cultured on different media supplemented with 2.5 μM Gibberellic acid. Germination was evoked in all the media in the presence of Gibberellic acid. Although the above protocol was repeated for embling development and field transfer but no true leaf emergence was recorded even after 12 weeks of field transfer.

The Main Advantages of the Present Invention are:

1) A reproducible and defined method for mass scale germination of cultured P. hexandrum embryos which can also work in other related species like Podophyllum peltatum

2) Light is not required for germination of the embryos and thus this increases the cost effectiveness.

3) This is the first defined method in which the light and dark requirements under in vitro conditions have been specified in terms of embryo germination, radicle emergence, hypocotyl elongation and cotyledon expansion along with growth of the healthy emblings.

4) This is the first defined method for circumventing the mechanical removal of dormancy during germination and true leaf emergence thereby ensuring mass scale propagation of plants.

5) The invention ensures successful field establishment coupled with very low i—mortality rate.

6) The invention expedites the process of true leaf emergence to the extent of 63% within 12 weeks

7) This invention by way of expediting the process of true leaf emergence ensures shortening the life cycle of plant by at least one season (8 months) as compared to what happens in nature. It also minimizes the time period for seedling establishment from 11 months to 4 years (Rust and Roth 1981, Singh et al., 1999) to 4-5 months only.

8) The present invention ensures statistically significant results for large-scale propagation of Podophyllum hexandrum.

9) The present method helps to domestication of plants in places other than their natural habitat

10) The present method helps to raise heterogeneous population of Podophyllum hexandrum Royle as to maintain the maximum genetic diversity which may be below the species level.

11) The present method ensures the transfer of ex situ raised plants to in situ conditions.

12) The present method ensures to provide a uniform protocol for field transfer of in vitro raised Podophlyllum hexandrum plants.

13) The present method ensures to specify nutritional requirement for Podophyllum hexandrum propagation under in vitro conditions.

14) The present method specifies the potting mix required at various stages of field transfer.

15) A producible and defined method for mass scale germination of cultured P. hexandrum embryos as compared to the above reports.

16) The method ensures successful field establishment coupled with very low mortality rate.

17) The method expedites the process of true leaf emergence to the extent of 63% within 90 days. This is in contrast to the above reports wherein no true leaf emergence is achieved.

18) This method by way of expediting the process of true leaf emergence ensures shortening the life cycle of plant by at least one season (8 months) as compared to what happens in nature.

1. An improved method for mass propagation of Podophyllum hexandrum Royle using embryo culture technique, said method comprising the steps of:
(a) placing an embryogenic tissue in a culture initiation medium comprising B-5 vitamins, supplemented with sucrose and agar and having pH of 5.8, at a temperature between 20 to 40° C. and subjected to a light/dark regime for about one week, to obtain embryos with radicals and expanded cotyledons,
(b) transferring the embryos to a basal MS medium supplemented with vitamins in the presence of about 14 hours of white fluorescent light of intensity of 52 μmol m⁻² s⁻¹, 10 hr dark period for 4 weeks to obtain emblings with well developed root, hypocotyl and green cotyledonary leaves,
(c) growing the emblings to acclamatize the plantlets for one week in bottles containing sand-soil and farm yard manure mix, B5 vitamins, without sucrose at a temperature between 20 to 40° C., light-dark regime and photo-period with light of intensity of 52 μmol m⁻² s⁻¹, and
(d) transferring the acclimatized plantlets to field to obtain healthy young plants with true leaves in 12 weeks.

2. A method as claimed in claim 1, wherein the embryogenic tissue used in step (a) is obtained from zygotic embryos which are cultured on basal B-S (Gamborg et al., 1968) medium supplemented with 3% sucrose.

3. A method as claimed in claim 1, wherein the light/dark regime of step (a) comprises exposure of the embryo to 14 hours of light and 10 hours of dark period.

4. A method as claimed in claim 1, wherein light is of intensity of 52 μmolm⁻²s⁻¹.

5. A method as claimed in claim 1, wherein the ratio of sand: soil: farm yard manure in step (c) is 3:1:1.

6. A method as claimed in 1 wherein BS medium was selected as the most suitable medium out of a range of media like B5, MS and WPM with their full and half strengths.

7. A method as claimed in claim 6, wherein B5 medium preferably comprises:

| | |
|---|---|
| Major Elements | |
| CaCl₂.2H₂O | 150 mg/litre |
| KNO₃ | 2500 mg/litre |
| MgSO₄.7H₂O | 250 mg/litre |
| (NH₄)₂SO₄ | 134 mg/litre |
| NaH₂PO₄.2H₂O | 150 mg/litre |
| Minor Elements | |
| KI | .75 mg/litre |
| H₃BO₃ | 3.0 mg/litre |
| MnSO₄.2H₂O | 10.0 mg/litre |
| ZnSO₄.7H₂O | 2.0 mg/litre |
| Na₂MoO₄.2H₂O | 0.25 mg/litre |
| CuSO₄.5H₂O | 0.025 mg/litre |
| CoCl₂.6H₂O | 0.025 mg/litre |
| Na₂EDTA | 37.3 mg/litre |
| FeSO₄.7H₂O | 27.8 mg/litre |
| Vitamins | |
| Inositol | 100 mg/litre |
| Thymine HCl | 2 mg/litre |
| Glycine | 10 mg/litre |

8. A method as claimed in claim 6, wherein MS and WPM media preferably comprise:

| MS Medium Composition | | WPM: Medium Composition | |
|---|---|---|---|
| Major Elements | | Major Elements | |
| NH₄NO₃ | 1650 mg/litre | NH₄NO₃ | 400 mg/litre |
| KNO₃ | 1900 mg/litre | Ca(NO₃)₂.4H₂O | 556 mg/litre |
| CaCl₂.2H₂O | 440 mg/litre | K₂SO₄ | 990 mg/litre |
| MgSO₄.7H₂O | 370 mg/litre | MgSO₄.7H₂O | 370 mg/litre |
| KH₂PO₄ | 170 mg/litre | CaCl₂.2H₂O | 96 mg/litre |
| Minor Elements | | KH₂PO₄ | 170 mg/litre |
| Na₂MoO₄.2H₂O | 0.25 mg/litre | Minor Elements | |
| H₃BO₃ | 6.2 mg/litre | ZnSO₄.7H₂O | 8.6 mg/litre |
| CoCl₂.5H₂O | 0.025 mg/litre | MnSO₄.2H₂O | 22.3 mg/litre |
| CuSO₄.5H₂O | 0.025 mg/litre | CuSO₄.5H₂O | 0.25 mg/litre |
| ZnSO₄.7H₂O | 8.6 mg/litre | H₃BO₃ | 6.2 mg/litre |
| MnSO₄.2H₂O | 22.3 mg/litre | Na₂MoO₄ | 0.25 mg/litre |
| KI | 0.83 mg/litre | Na₂EDTA | 37.3 mg/litre |
| Na₂EDTA | 37.25 mg/litre | FeSO₄.7H₂O | 27.8 mg/litre |
| FeSO₄.7H₂O | 27.85 mg/litre | Vitamins | |
| Vitamins | | Inositol | 100 mg/litre |
| Glycine | 2 mg/litre | Threonine HCl | 1 mg/litre |
| Inositol | 100 mg/litre | Nicotinic Acid | 0.5 mg/litre |
| Thymine HCl | 1 mg/litre | Pyridoxine HCl | 0.5 mg/litre |
| Nicotinic Acid | 0.5 mg/litre | Glycine | 0.2 mg/litre |
| Pyridoxine HCl | 0.5 mg/litre | | |

9. A method as claimed in 1 wherein culturing of the excised zygotic embryos on B5 medium under dark conditions at 25±2° C. for one week and transfer of the emblings with radicle, hypocotyl and expanded cotyledons after 2 weeks to light/dark photoperiod regime (14h light/10h dark).

10. A method as claimed in 1 wherein the composition of potting mix has been specified to be 3:1 of sand :soil with 0.6N B5 medium without sucrose and a mix of sand: soil:farm yard manure in the ratio of 3:1:1 for Pododphylluim plant growth.

11. A method as claimed in 1 wherein the green house conditions for successful plant establishment and true leaf emergence was selected out of the use of poly tunnels covered with and without green agro net and planting in Hikkotrays, pots and directly into potting mixes in dug trenches.

12. A method as claimed in 1 wherein the ex situ raised plants can be transferred to their natural habitat

13. A method as claimed in 1, wherein the heterogeneous population of Pododphyllum can be raised to conserve genetic diversity, which may be below level of species.

14. An improved method for mass propagation of Podophyllum hexandrum Royle using embryo culture technique, said method comprising:
(a) collecting mature pods of P. hexandrum Royle from their natural habitats in the Western Himalayas, India at an altitude ranging from 2700 m-3500 m above mean sea level during the last week of August and first week of September,
(b) separating the seeds from the pulp immediately after collection, prior to disinfection with 0.1% Bavistin and streptomycin sulphate (0.1%) for 15 minutes followed by a thorough washing of seeds after about 1 minute dip in 70% alcohol and surface sterilization in 0.1% mercuric chloride for 7 minutes, all traces of mercuric chloride were finally washed off by 4-5 rinses in sterile distilled water,
(c) dissecting the surface sterilized seeds by a sharp excision with a scalpel blade at the base of the microplyar lobe and embryos were carefully squeezed out of their micropylar groove,
(d) culturing the embryos at 25±2° C. in dark on basal B-5 (Gamborg et al., 1968) medium supplemented with 3% sucrose of pH 5.8 adjusted with 1N KOH and HCl prior to the addition of agar (0.8%) and autoclaved at 121° C.,
(e) transferring the emblings with fully developed radicals and expanded cotyledons after 2 weeks to about 14 hour photoperiod with light of intensity of 52 ptmolm²sI alternating with 10-h dark period for further growth,
(f) transferring of the 4 week old emblings (8.0 cms long) with well developed root, hypocotyl and green cotyledonary leaves to jam bottles (covered with lids) containing 120 g of sand: soil in the ratio 3:1 enriched with 0.6 N (liquid) of B5 medium without sucrose,
(g) hardening of the emblings for about 1 week under lab culture conditions (25±2° C.) and 14 h photoperiods with light intensity of 52 μmolm⁻²s⁻¹ alternating with 10-h dark period),
(h) transferring the jam bottles containing the emblings to green house for another one week with partially opened lids,
(i) transferring of at least 8.0 cm long emblings to 4′ pots containing potting mix of sand: soil: farm yard manure in the ratio: 3:1:1 covered with magenta jars for 6 weeks,
(j) removing the magenta jars from over the plants after about 6 weeks as true leaves start emerging,
(k) growing the plants again for about 6 weeks for further emergence of true leaves, and
(l) transferring the healthy plants to natural conditions after 6 months.
